(19) United States
(12) Patent Application Publication
(10) Pub. No.: US 2011/0287407 A1
(43) Pub. Date: Nov. 24, 2011

(54) INTEGRATED METHODS AND SYSTEMS FOR PROCESSING A MOLECULAR PROFILE

(75) Inventor: Frederic Zenhausern, Fountain Hills, AZ (US)

(73) Assignee: DIOMICS CORPORATION, La Jolla, CA (US)

(21) Appl. No.: 13/095,842

(22) Filed: Apr. 27, 2011

Related U.S. Application Data
(60) Provisional application No. 61/343,337, filed on Apr. 27, 2010.

(51) Int. Cl. G01N 35/00; C12Q 1/02; G06F 19/10; G06F 17/18; C12Q 1/70; C12M 1/34; B82Y 15/00

(52) U.S. Cl. 435/5; 436/43; 435/29; 435/287.1; 422/63; 702/19; 977/742; 977/762; 977/902

(57) ABSTRACT

An automated and integrated matching of a biological specimen to an individual, can include a collector having substrate materials and configured to selectively collect and release at least one bio-molecular species from a biological specimen; a self-contained mobile automated testing instrument configured to receive the biological specimen and further configured to generate, store and output a molecular profile of the at least one predetermined bio-molecular species; a processor based system communicatively coupled to the self-contained mobile automated testing instrument and configured to receive the molecular profile; and wherein the processor based system is configured to provide a quantitative comparative analysis and report of the molecular profile with a selected molecular profile. In one approach, at least one predetermined bio-molecular species and stored pre-selected molecular profile is for a predetermined sequence of interest, such as a DNA-profile as defined by a Combined DNA Index System (CODIS) system.

### CROSS-REFERENCE TO RELATED APPLICATION

This application claims priority to U.S. Patent Appl. No. 61/343,337, filed Apr. 27, 2010, which is incorporated herein by reference in its entirety.

### FIELD OF THE INVENTION

Methods and systems for processing a molecular profile are described herein and, in particular, methods and systems for processing the molecular profile of a specimen collected from an organism.

### BACKGROUND

Latest advances in personalized medicine promise benefit to thousands of patients. For example, under current medical practice standards, people in an early disease stage (e.g., early stage cancer) can undergo series of clinical diagnostic tests, such as from medical imaging procedures and from collected body fluids and tissues. Analyzed test results can be used to assist a health care provider and patient with decisions related to treatment options, such as surgery (e.g., removal of a tumor), followed by observation, and medication, if applicable.

Improved and more personalized medicine can also be enabled by a combination of techniques from biotechnology, bioinformatics and genomics. The practice of medicine using genetically based diagnostic tests and targeted pharmacologic treatment can provide pre-emptive strategies beyond the prevention of diseases. Examples can include the detection of a predisposition to a disease.

### SUMMARY

Accordingly, the embodiments presented herein can provide methods and systems for processing a molecular profile are described herein and, in particular, methods and systems for processing the molecular profile of a specimen collected from an organism.

In one approach, the embodiments can include a method for automated processing and analysis of a specimen from an organism, the method comprising receiving a biological specimen into an automated and integrated micro-device instrument, the biological specimen released from a collector that selectively collects species from the biological specimen; isolating at least one molecular species representing at least one set of genetic characters of the specimen; supplying at least one reagent to perform an assay on the at least one molecular species to detect at least one representative expression phenomenon of genetic characters; monitoring at least one change in the at least one representative expression phenomenon during the assay; translating the genetic characters into a molecular profile; and analyzing the molecular profile using at least one of a statistical and mathematical algorithm to generate an information pattern that serves as an indication of a future biological event correlated to the genetic characters.

In one approach, the step of measuring the molecular data using a statistical algorithm can include the steps of mining databases for comparative molecular profiles; and comparing the molecular profile of the specimen with a comparative molecular profile from the database.

In another approach, a rapid diagnostic method for a human specimen, can comprise the steps of: providing a specimen to a mobile automated integrated device capable of analysis of specimen preparations having fluid volume as small as 1 fl, low target concentrations of as little as 1 fmol, and small features of as small as 1 nm; processing the specimen to prepare a target agent for at least one of microscopic, genetic, proteomics and genomic analysis; interrogating the target agent; collecting at least one of a morphological and molecular characteristic of the target agent; comparing the characteristic of the target agent with a reference agent; analyzing a difference between target and reference agents to provide information about at least one of a morphology, genome, proteome, and metabolome of the target agent; generating a data output; and outputting the data output to a computer via a communication network. In one approach, the specimen is a nucleic acid. In another, the integrated device comprises a microfluidic channel network. In some embodiments, the target can be a viral pathogen, a bacterial pathogen, and/or a spore (such as a anthracis species).

In some embodiments, an analysis can include a power light emitter, optical means to guide radiation from a radiation source and collect radiation from the target agent when interrogated by the source, a detector, and a device for microscopic analysis. The emitter can be from the family of materials selected from the list of carbon nanotubes, nanowires and carbene.

In some embodiments, the molecular characteristic of the target agent can be a genetic characteristic selected from one of a gene expression and a protein expression.

Another preferred embodiment can provide an automated and integrated matching of a biological specimen to an individual, comprising: a collector having substrate materials and configured to selectively collect and release at least one bio-molecular species from a biological specimen; a self-contained mobile automated testing instrument configured to receive the biological specimen and further configured to generate, store and output a molecular profile of the at least one predetermined bio-molecular species; a processor based system communicatively coupled to the self-contained mobile automated testing instrument and configured to receive the molecular profile; and wherein the processor based system is configured to provide a quantitative comparative analysis and report of the molecular profile with a selected molecular profile. In one approach, at least one predetermined bio-molecular species and stored pre-selected molecular profile is for a predetermined sequence of interest, such as a DNA-profile as defined by a Combined DNA Index System (CODIS) system. In one approach, the DNA-profile can be at least one allele at the 13 CODIS core loci for forensic STR DNA analysis.

In one embodiment, the processor based system can be further configured to provide a qualitative comparative analysis of the molecular profile output from the test instrument with a stored pre-selected qualitative criteria profile, and configured to output a feedback adjustment to the self-contained mobile automated testing instrument when a predetermined qualitative threshold is not achieved. Feedback adjustments can adjusts a bioassay process of the self-contained mobile automated testing instrument. The system can optionally include a memory of the processor based system, wherein the pre-selected molecular profile is stored on the memory and/or a third party database communicatively coupled to the processor based system wherein the pre-selected molecular profile is stored on the third party database.

Another preferred embodiment can provide a computer system to provide automated analysis of molecular profiles of biological specimens, the computer system comprising: an interface configured to receive a molecular profile of a biological specimen collected with a collector and interrogated to result in the molecular profile; at least one processor; at least one memory storing executable program instructions, wherein the processor is programmed, via execution of the executable program instructions, to: perform an analysis of the molecular profile relative to at least one comparative molecular profile retrieved from a database; and provide a report based on the analysis.

In one approach, the analysis comprises a quantitative comparative analysis. In another approach, the computer system analysis comprises a quantitative statistical comparative analysis.

In one approach, the processor can be programmed, via execution of the executable program instructions, to: determine database search parameters corresponding to the molecular profile: and retrieve the at least one comparative molecular profile from the database based on the database search parameters.

In one approach, the computer system processor can be programmed, via execution of the executable program instructions, to provide the report for display at a remote computer device.

In one approach, the computer system processor can be programmed, via execution of the executable program instructions, to output feedback adjustment signaling to an instrument configured to process the biological specimen as a part of the process of creating the molecular profile.

In one approach, the computer system processor can also include an analysis instrument configured to: process the biological specimen for detection; detect a genetic characteristic of the biological specimen; generate the molecular profile; and output the molecular profile to the interface of the processor. In one embodiment, the analysis instrument can be communicatively coupled to the interface of the processor and/or to the interface of the processor via a network.

In one embodiment, the processor can be configured to perform the analysis and provide the report in an automated manner upon receipt of the molecular profile.

Another preferred embodiment can provide a computer implemented method for automated analysis of molecular profiles of biological specimens, the method implemented with at least one processor executing program instructions stored in at least one memory, the method comprising; receiving, via a computer system interface, a molecular profile of a biological specimen collected with a collector and interrogated to result in the molecular profile; retrieving at least one comparative molecular profile from a database; performing, using the at least one processor, an analysis of the molecular profile relative to the at least one comparative molecular profile; and providing, using the at least one processor, a report based on the analysis.

Other features will become more apparent to persons having ordinary skill in the art to which the package pertains and from the following description and claims.

### BRIEF DESCRIPTION OF THE DRAWINGS

The above and other aspects, features and advantages of the present embodiments will be more apparent from the following more particular description thereof, presented in conjunction with the following figures, wherein:

FIG. 1 is a schematic overview of one embodiment of the present system and method to automate processing a molecular profile;

FIG. 2 is a system flow diagram in accordance with one embodiment of the method shown in FIG. 1;

FIG. 3 illustrates the system and network environment according to several of the present embodiments; and

FIG. 4 illustrates a simplified block diagram of a processor-based system for implementing methods described according to one or more embodiments.

Corresponding reference characters indicate corresponding components throughout the several views of the drawings.

### DETAILED DESCRIPTION

The following description is not to be taken in a limiting sense, but is made merely for the purpose of describing the general principles of exemplary embodiments. The scope of the invention should be determined with reference to the claims. The present embodiments allow improved human identification such as DNA forensic analysis. In particular, the present embodiments provide necessary components and processes to preferably integrate DNA fingerprinting. An integration of workflow processing can provide minimal contamination and environmental interference while automation can preferably reduce the consumption of reagents, minimize sample volume to be interrogated while possibly reducing the cycle time from sample collection to data analysis and decision-making when related to, for example, a human identification. Other applications can related to humanitarian intervention, medical and/or chemical or biological countermeasures for disaster response or monitoring of a radiological event or a nuclear accident, response to a terrorist attack or any related event of relevance to national security or public health emergency, and the like.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Despite advances in the art, improvements in health care delivery and forensics identification are possible and desired. Such improvements could include faster and more efficient testing, analysis, and results reporting. As an added benefit, these types of improvements could potentially also be applied to other areas of science and medicine such as screening tests, forensics identification, accessing individuals risk to a phenotype, infectious diseases, sequencing (e.g. nucleic acids such as DNA or RNA), public health emergencies, disaster response and preparedness, molecular diagnostics, disease predisposition and sensitivity or any related biomolecular assay test known in the prior art, including protein, metabolite or its at least one of its by-product monitoring or any molecule in any phase, i.e. liquid, solid or gas, which may be acting or having a role as a marker of a biological reaction or event, either at the molecular, cellular or tissue or even at the organism level, or, in the case of a human species, as an individual or person, Cancer, cardiovascular and neurological diseases, genome wide association studies, proteomics, metabolomics, genomics or other -omics testing, and the like. Accordingly, methods and systems for processing a molecular profile are described herein and, in particular, methods and systems for processing the molecular profile of a biological specimen collected from an organism automated and integrated processing of a molecular sample to provide molecular profiling, analysis, and reporting that can be remote and in real time.

Some embodiments provide methods and systems for integrated and automated processing of a biological specimen such as a fluid (e.g., blood, saliva, semen, and the like) or tissue sample collected from an organism, such as a person, and its molecular interrogation. Testing types can be varied, but can include, by way of example, biopsy, molecular diagnostics, imaging screening, and the like to generate useful information, such as information to assist in a decision for a medical intervention targeted to the individual. Such intervention decisions can also factor, by way of example, risks preferably defined through genetics, environment, clinical and family histories, and the like. Interventions could include diagnostics, disease prognosis, therapy (immune, hormonal, chemo, risk of drug resistance, etc) and other medical procedures or personal care that could benefit the health of an individual. It is also contemplated that the embodiments presented herein can also assist in other scientific disciplines, such as agricultural (i.e. animal or plant identification), human identification in forensics application, bacterial or viral identification for medical or environmental purposes (e.g. pandemic infection, water treatment), but also any system in which a molecule or part thereof may be combined in a coding mode for tracking a biospecimen but also a manufactured article (e.g. pharmaceutical biologics, an artwork, an electronic component or appliance, and the like. In one embodiment, the method and system can process at least a set of molecular species that can have some genetic characteristics of a subject, and providing a statistical analysis of the molecular profile to generate information for tailoring a personalized medical procedure or identifying an individual.

The following definitions are provided to assist in understanding some embodiments of the present methods and systems. Unless specifically defined otherwise, all technical and scientific terms used herein have the same meaning as commonly understood by one of ordinary skill in the art of the present embodiments. Generally, the nomenclature used herein and the laboratory procedures in cell culture, chemistry, microbiology, molecular biology, cell science and cell culture described below are well known and commonly employed in the art. Unless specifically described otherwise, conventional methods are used for the testing procedures, such as those provided in the art. Where a term is provided in the singular, the plural of that term should also be contemplated. The nomenclature used herein and general laboratory procedures described herein are known and commonly employed in the art. As employed throughout the disclosure, the following terms, unless otherwise indicated, shall be understood to have the following meanings:

“Organism” can be any prokaryote or eukaryote, and can include viruses, bacteria, plants, yeast, protozoans, and metazoans. Metazoans include vertebrates and invertebrates. “Organism” can also ref to more than one species that are found in association with one another, such as mycoplasm-infected cells, a plasmodium-infected animal, etc.

A “nucleic acid molecule” is a polynucleotide. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases such as 2-aminoadenine and the like. A nucleic acid molecule can have linkages other than phosphodiester linkages. A nucleic acid molecule can also be a peptide nucleic acid molecule. A nucleic acid molecule can be of any length, and can be single-stranded or double-stranded, or partially single-stranded and partially double-stranded. Other “molecular species” may comprise lipids, proteins, carbohydrates, small molecules such as hydrocarbons, aldehydes, ketones or other part thereof, and present in multiple phases such as gas, liquid or solid.

A “detectable label” is a compound or molecule that can be detected, or that can generate a readout, such as an electromagnetic wave, fluorescence, radioactivity, color, chemiluminescence or other readouts known in the art or later developed.

“Sequence identity” or “identical” means that two polynucleotide sequences are identical (for example, on a nucleotide-by-nucleotide basis) over the window of comparison. “Partial sequence identity” or “partial identity” means that a portion of the sequence of a nucleic acid molecule is identical to at least a portion of the sequence of another nucleic acid molecule.

“Substantial identity” or “substantially identical” as used herein denotes a characteristic of a polynucleotide sequence, wherein the polynucleotide comprises a sequence that has at least 30 percent sequence identity, preferably at least 50 to 60 percent sequence identity, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison. “Substantial partial sequence identity” or “substantially partially identical” is used when a portion of a nucleic acid molecule is substantially identical to at least a portion of another nucleic acid molecule. As used herein “identity” or “identical” refers to the base composition of nucleic acids, and not to the composition of other components, such as the backbone that can be comprised of one or more sugars and one or more phosphates, or can have other substituted moieties.

A “gene” normally exists as two strings of nucleotides entwined in a helical shape resembling a spiral staircase. There are four DNA nucleotides, adenine (A), cytosine (C), guanine (G) and thymine (T), and the sequence of these nucleotides in a gene encodes the information a cell uses to build a specific protein.

A “mutation” is a change in the genome with respect to the standard wild-type sequence. Mutations can be deletions, insertions, or rearrangements of nucleic acid sequences at a position in the genome, or they can be single base changes at a position in the genome, referred to as “point mutations”. Mutations can be inherited, or they can occur in one or more cells during the lifespan of an individual.

A “sequence of interest” is a sequence whose presence or variation can be detected in one or more survey populations of nucleic acids.

A “single nucleotide polymorphism” or “SNP” is a position in a nucleic acid sequence that differs in base composition in nucleic acids isolated from different individuals of the same species.

A “solid support” is a solid material having a surface for attachment of molecules, compounds, cells, or other entities. The surface of a solid support can be flat or not flat. A solid support can be porous or non-porous. A solid support can be a chip or array that comprises a surface, and that may comprise glass, silicon, nylon, polymers, plastics, ceramics, or metals. A solid support can also be a membrane, such as a nylon, nitrocellulose, or polymeric membrane, or a plate or dish and can be comprised of glass, ceramics, metals, or plastics, such as, for example, a 96-well plate made of, for example, polystyrene, polypropylene, polycarbonate, or polyallomer. A solid support can also be a bead or particle of any shape, and is preferably spherical or nearly spherical, and preferably a bead or particle has a diameter or maximum width of 1 millimeter or less, more preferably of between 0.5 to 100 microns. Such particles or beads can be comprised of any suitable material, such as glass or ceramics, and/or one or more polymers, such as, for example, nylon, polytetrafluoroethylene, TEFLON™, polystyrene, polyacrylamide, sepaharose, agarose, cellulose, cellulose derivatives, or dextran, and/or can comprise metals, particularly paramagnetic metals, such as iron. (See also generally, U.S. Pat. No. 6,900,013 to Wang, et al.)

Four classes of biological molecules are known, namely, those having proteins, lipids, carbohydrates and nucleic acids. Of interest herein, are the nucleic acid molecules namely, DNA, a genetic component of all cells, and RNA, which usually functions in a synthesis of proteins. Although building blocks of information have been identified as representative of the life of an individual that can comprise DNA, RNA, chromosomes, alleles, single nucleotides polymorphisms (SNP's), genetic markers responsible for specific disease states are often pulled from biological materials such as DNA or RNA.

The present embodiments can be practiced with many types of molecules, but are preferable biomolecules. By way of example only, the present embodiments are described for biomolecules having DNA. DNA is emphasized because it is the prime genetic material, carrying all hereditary information within chromosomes. In this context genetic characters are also referring to a first level of expression of genes or other biological reaction representative of the presence and/or concentration or density of a species or mutation, or deletion of a component of genetic materials that can result in a change of a second biological response such as phenotypes.

Personalized medicine is an emerging trend in treatment strategies that can incorporate more individually, genetically selected approaches to develop therapies that would be safer and more effective. For example, better health outcomes are possible where pharmacogenomic products can be prescribed to the patients who are genetically pre-disposed to respond. Pharmacogenomics through sequencing and screening technologies is also able to define precise disease types. At the core of better diagnostics and prognosis of a disease is the information contained in the genes of an individual in sequences of interest.

Molecular diagnostics is becoming increasingly linked with the detection and identification of a multitude of pathogens that can be processed for generating a pattern of information or bio-signature that can represent a disease state and/or type. Electron microscopic diagnosis is uniquely suited for rapid identification of infectious agents. A specimen can be ready for examination and an experienced technologist can identify, by electron microscopy, a viral or bacterial pathogen morphologically within 10 minutes of arrival in the electron microscopy laboratory. In the art, the role of transmission electron microscopy in viral or bacterial diagnosis and outbreak management; methods for specimen collection, preparation and examination; laboratory safety and quality control; and the differential morphologic diagnosis of infectious agents are known. More recently though, gene-expression and proteomics-based assays can demonstrate high performance in detecting biological phenomena and related signaling pathways related to diseases. The present embodiments relate to an integrated approach for the specimen collection in gene-expression and proteomics-based assays, including preparation and interrogation.

In a preferred embodiment, a system platform can have several components to process various steps of a sample collection, sample preparation within a nano-scale and/or micro-scale. For example, the device would be able to analyze sample preparations than can include using small volume of fluids (e.g., at little as about 1 fl), low target concentrations (e.g., at as little as about 1 fmol), and small features (e.g., down to about 1 nm) for containing, moving, supporting (e.g., solid support or substrate surface in the case of an array), controlling, interrogating and characterizing the sample specimen, or a part thereof. In this instance the device would preferably have a microfluidic cartridge comprised of modules including channel network, pumps, valves, sensors and actuators. For example, lab automated systems (e.g., Evo system from Tecan, ABI/Lifetech CE 3130 or 9500 system, or other commercially available system, such as the Genbench system from Network Biosystems or the Apollo 200 from IntegenX) could provide some modules for some embodiments of a lab automation component. In other embodiments, a more complete integrated system as the Midas system included in the literature reference may provide a more integrated approach. (See also, U.S. Pub. No. 2010/0144558 to Zenhausern et al. entitled Systems and Methods for Biodosimetry with Biochip Using Gene Expression Signature, the specification and figures of which are incorporated herein by reference.) Accurate clinical diagnostics, and forensic studies, often hinge on the collection and separation of biological samples. A common reason to collect these samples is to extract DNA for profiling and identification.

Some embodiments provide a fully integrated method for profiling a bio-specimen, such as from a human individual. For example, in one approach, the specimen can be processed within an analysis instrument to prepare a target agent or sample of the specimen for at least one of a microscopic, genetic, proteomics and/or genomic analysis. The target agent can be interrogated within an area of said device where the analysis is performed. Such interrogation may be performed by a sample detector, the detector including one or types of interrogation devices. Exemplary target agents can also include proteins, viral pathogens, bacterial pathogens, spores (e.g., from an anthracis species), infectious disease vectors (e.g., insects), and the like.

During this analysis, the system can collect at least one of a morphological and/or molecular characteristic of the target agent, such as a “sequence of interest”; compare the characteristic of the target agent with a reference agent; analyze a difference between target and reference agents to provide information about at least one of a morphology, genome, proteome, and/or metabolome of the target agent to generate a data output; and process the device data output to a computer via a communication network.

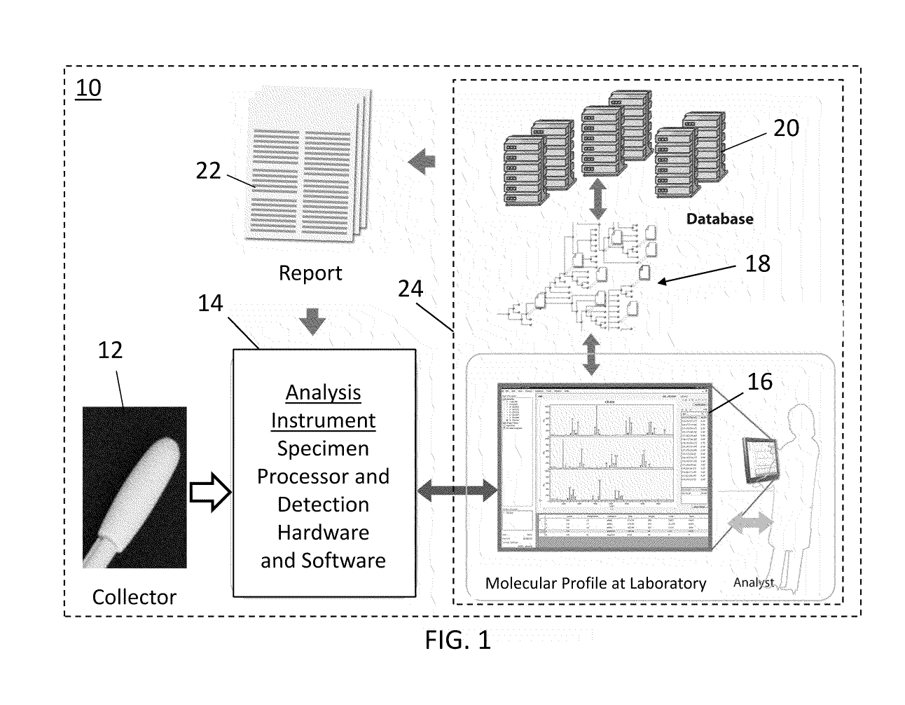

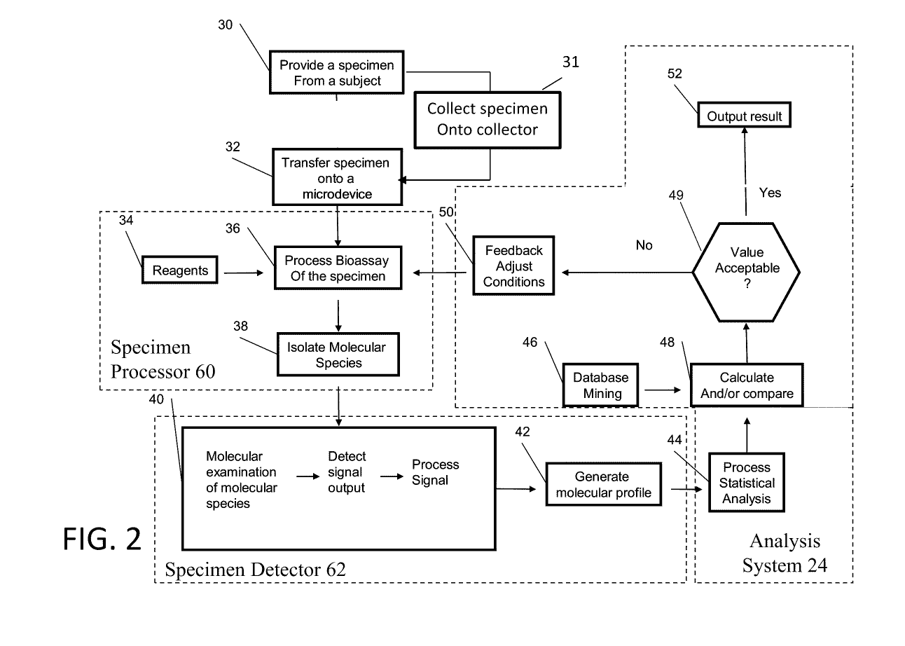

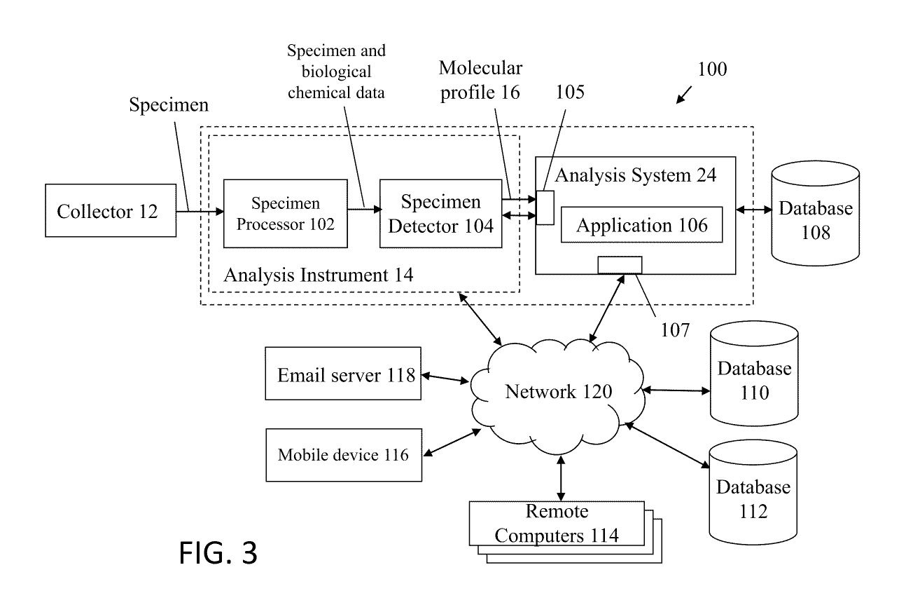

Turning now to the figures, FIG. 1 illustrates a schematic overview of a process flow of one embodiment of the present system and method to automate processing a molecular profile of an organism, which is generally indicated as system 10. The system 10 as illustrated generally combines profiling a set of biological signatures using a specimen collector, an integrated processing and detection device (generically referred to herein as an analysis instrument 14, and in some embodiments, using a microfluidic-device) for preparing the specimen or sample, and a molecular analysis system 24 that performs a molecular analysis of the specimen/sample and data processing using an analysis algorithm (e.g., a statistical analysis algorithm) to interpret a molecular profile of a subject for personalized medicine or other indicated uses. FIG. 2 illustrates one embodiment of a flow diagram for the system of FIG. 1. FIG. 3 is a schematic diagram of a network environment that can be implemented for the embodiment of FIG. 1 allowing all parts of system 10 to be communicatively connected.

The general components of system 10, as shown in FIG. 1, can include a collector 12 to collect a specimen (or sample), and an automated analysis instrument 14 (also referred to herein as the analysis instrument 14) to analyze the specimen and generate a molecular profile 16 of the specimen. By way of example, the analysis instrument 14 includes specimen processor and specimen detection/interrogation hardware and software. For example, in one embodiment, the specimen processor includes all hardware and applicable software to prepare the specimen for detection/interrogation and the specimen detection/interrogation includes all hardware and applicable software to analyze the prepared specimen for example, using at least a microscopic, genetic, proteomics and/or genomic analysis of the specimen. In some embodiments, the detection portion of the analysis instrument 14 outputs a molecular profile 16 of the interrogated specimen. The system 10 also includes a molecular analysis system 24 (also referred to herein as analysis system 24) coupled to the analysis instrument 14 and to a database 20. In some embodiments, the analysis system 24 is a processor-based system having at least one microprocessor, at least one memory device or computer readable medium, and executable program instructions stored by the at least one memory device or computer readable medium and executed by the at least one microprocessor to perform one or more functions, such as those described herein. In some embodiments, the analysis system 24 can access, search and retrieve (e.g., using search algorithm that is part of an analysis algorithm 18) one or more comparative molecular profiles from the database 20. The analysis system 24 can compare these comparative molecular profile/s to the molecular profile 16 generated by the analysis instrument 14, e.g., the molecular profile 16 may include electroferrogram data, microarray image/s, and/or any other known data points or information. In some embodiments, an analysis of the comparison of the sample and comparative molecular profiles by the analysis algorithm 18 can be generated as a report 22. The contents of the report 22 may comprise literature, report, text, data points, level of matching, level of correlation, scoring, event or condition correlation or prediction information, quality assurance indicator, or any other known reporting information and may be a function of the specific molecular analysis being performed.

Collector 12 can be any type of collection device used to dynamically (e.g., there is a responsive interaction like and active feedback to actually select a predetermined species) collect a biological specimen from a living or non-living subject from the subject or from surfaces previously in contact by the subject. For example, in one embodiment, collector 12 is a simple cotton swab or similar device. In other embodiments, the collector 12 may include novel approaches to collect a bio-specimen that can preserve sample integrity for subsequent molecular analysis. For example, the specimen can be preserved for testing by from about a few days, and preferably about up to several years. Generally, preservation methods can include any variety or combination of specimen encapsulation, embedding in a solid matrix, such as alginate, micro beads, preservative reagents, and the like. Also, temperature affects, elution timing, and assays to process the specimen can be considered. The specimen could also be preserved in solid phase matrices such as the ones commercially available from manufacturers as GE, QChip, Biometrica or Promega. Note that preservation could also be obtained by using saturated solution of some biomolecular reagents stored in constrained microenvironment (e.g., a microfluidic cartridge) or in a container (e.g., containers manufactured by Pharmacia) prior to be delivered for the assay reaction. Collector 12 can be configured to collect a biospecimen from an organism and provide physical-chemical properties and scaffold for sorting desired biological species from the specimen based on physical size and/or shape but also based on predetermined chemical selectivity or other specific attribute. In other words, in some embodiments, collector 12 can include substrate materials to provide a scaffold to selectively collect predetermined and desired species from said biological specimen. Also, in some embodiments, collector 12 can have soluble components to allow for rapid release of the specimen. In these embodiments, such components should not affect the desired species of the specimen such as allowing it to break down, or change morphology in any way.

By way of example, in one approach, collector 12 can exploit alternative high surface area materials that could replace known collection devices such as cotton swabs that are typically used in the clinics, at a laboratory or in the field. In some cases, high surface area is important to maximize bonding opportunities of the biological samples or specimens to the collector. A targeted feature can be that, unlike cotton, the collected samples can be efficiently removed from the collector by a wash or, in the spirit of the enzyme digestion of cotton fibers, the collector will be completely broken down and will not interfere with further separation or extraction processes. Current properties of interest include hydrophilic/hydrophobic and even chemotaxis and/or durotaxis possibilities. Coblocked polymers crosslinked aerogels (manufactured material with the lowest bulk density of any known porous solid) are examples of ideal materials because of low production costs, high surface area, and the extensive property manipulation that can be obtained by altering the polymer contents of the aerogel (x-aerogel).

Collector 12 can also be formulated novel inorganic-organic hybrid composite materials that have incorporated into their structure active molecules with targeted therapeutic properties, such as antibiotic activity by the inclusion of a known antibiotic such as gentamicin or nano-sized silver ions (Ag(I) or Ag(0)). The inorganic-organic hybrid composite (e.g. Si₂PEOx) can be synthesized by sol-gel methods. (See generally the subject matter of, WIPO International Publication No. WO 2010/019920 to Zenhausern et al. and entitled “Porous Materials for Biological Sample Collection”, which is incorporated herein in its entirety by reference.) Physical properties of such a hybrid composite can further be tuned and/or enhanced by the addition of additional Si—O and/or Ti—O bonds into the inorganic-organic hybrid composite to modify the mechanical properties of the materials, for example. The additional Si—O and Ti—O particles can be added to the inorganic-organic hybrid composite by reacting tetraethoxysilicate (TEOS) and/or titanium isopropoxide during the hydrolysis of the inorganic-organic hybrid composite. The resulting structure can have nano-domains of either silica or titanium nanoparticles connected together by PEO or other organic molecules. Alternative nanoparticles can be added to the polymer to provide magnetic effects that can then be exploited with the formulation of reagents for preparing magnetic based extraction chemistries to isolate nucleic acids, for example.

In some embodiments, the analysis instrument 14 can interface with collector 12 and be a fully automated apparatus for molecular analysis. In some embodiments collector 12 can optionally use a cartridge compatible to be interfaced and received by the instrument, which would allow for automatic and specimen retrieval. Note the collector could be either in a format suitable for direct dissolution into an inlet of a cartridge or it could be part of a fluidic module to be interface with a cartridge, similarly to an automated liquid injector used in lab automated systems. In some embodiments, instrument 14 can have other automated features such as time keeping devices, nucleic acid amplification capabilities (i.e. PCR, LCR, etc), and a geographical positioning means, using for example, global positioning satellites, or coordinated radio frequencies. In this instance the specimen could be recorded and reported in a system database (e.g., a database local to or remote from the instrument 14) as time and location of specimen collection. In some embodiments, this data can be used to compare against environmental stressors or predictors in the specimen analysis. Further, geographical location can be used to adjust system testing and reporting parameters. For example, variations in reportable data among countries can be accounted for.

Preferably, analysis instrument 14 can isolate at least one predetermined molecular species representing at least a set of genetic characters of the specimen. Also, analysis instrument 14 is preferably an automated and portable device to allow it to be brought into the field where the specimen is collected. Accordingly, in these embodiments, instrument 14 should be robust in construction to allow minimal chance for specimen contamination and mobility for rapid analysis of specimens, e.g., at a crime scene or other site of specimen collection. These advantages, such as of the mobility of instrument 14, can be improved by accessing and/or interfacing with databases immediately after profile generation, etc. Further, instrument 14 should allow for fast and rapid retrieval of the predetermined species from the collector. Therefore, the assay, reagents, and collector is preferably allows for rapid solubility. Furthermore, in some embodiments, the analysis instrument 14 is not a mobile device but is integrated with the analysis system 24 at a laboratory or other location.

In some embodiments to provide forensics identification based on a human specimen, analysis instrument 14 uses rapid DNA testing instrumentation such as performing an Short Tandem Repeat (STR) assay. Over the last decade, STR genotyping analysis has been the most commonly used technique in forensic medicine, including human identity testing, missing person investigations, and identification of victims of mass disaster. Recently STR profiling has also been emerging for non-forensic surgical tissue identification, fetal DNA identification from maternal plasma, and authentication of human cell lines, stem cells and tissues, due to its capability of higher analytical resolution while using small amounts of DNA sample. The overall analysis protocol integrates the number and types of selected loci, the data quality control, the results interpretation and building of a national STR database, which may also be implemented as a standard for biospecimen identification. More recently, a prior art report listed 360 cross contaminated human cell lines, particularly for cancer, suggesting performing routine identification testing is critical for biobanks.

Currently, a genetic identity test is sometimes carried out in the clinical laboratories or surgical pathology department by staff technologists, often manually on the bench setting, which may be a potential source of cross-contamination. In one embodiment of the present system, a fully integrated “sample-to-answer” STR is provided profiling microfluidic system for biospecimen identification. This proposed “point of contact” system can derive from an instrument currently under DNA forensics validation by international police forces, and perform automated STR genotyping analysis of tissue biospecimen. The automation of the sample preparation workflow can be performed using an automated miniaturized fluidic cartridge, whose single-use and disposable configuration, with self-contained actuators and reagent packaging onto a plastic substrate to minimize contamination issues. In some embodiments, the analysis instrument 14 interfaces with user-friendly data acquisition and automated data analysis features (e.g., implemented by the analysis system 24) to complement the system 10 into a cost-effective, highly sensitive and rapid DNA fingerprint platform for point-of-care application. A purified DNA sample can also be archived within the cartridge for long term storage and future retrieval for downstream molecular analysis. This rapid STR system (raSTR) could be readily implemented in a clinical setting and provide a valuable tool to address specimen mislabeling, un-labeled sample identification, clinical sample misidentification, and tissue contamination characterization.

An interface of collector 12 to the analysis instrument 14 can include the automated steps of dissolution of the collector substrate into materials components that are soluble into fluids for processing with the apparatus and preferably suitable for compatible reaction with assay chemistries for the extraction of genetic materials and, preferably, DNA or RNA. After receiving the specimen, in some embodiments, the analysis instrument 14 introduces one or more reagent(s) for performing a biological assay on the molecular species to detect at least one representative expression phenomenon (by way of specific example only, in some embodiments, the representative expression phenomenon may include at least one phenotypic change) of genetic characters of the specimen. Instrument 14 can then monitor for at least one change in the expression phenomenon during the assay to translate the genetic characters into a molecular data profile 16. It is noted though that there are many possible types of expression phenomenon that can be considered within the scope of the present embodiments. Such expressions can include, but not limited to, gene expression, mutations, deletions, chromosome aberrations, micronuclei, or other ribosomal or nuclear alterations including post-translational methylation, and the like. The resultant molecular profile 16 can be described in many ways within the scope of the present embodiments based on the detection mechanism of the instrument 14 and can include, by way of example only, impedance, optical characteristics, mass spectrometry, including gas chromatography-mass spectrometry (GC-MS), capillary electrophoresis.

In some embodiments, analysis by instrument 14 can optionally have a power light emitter, optical means to guide radiation from a radiation source and collect radiation from the target agent when interrogated by the source, a detector, and a device for microscopic analysis. A preferred embodiment comprises epifluorescence detection system, Raman scattering spectroscopy, near-field optics or any related optical detection scheme practiced in the prior art. For example, an optical system exploiting integrated optics may be preferably implemented. (See generally, Zenhausern et al. US Patent Pub. No's: 2010/0221726 and 2010/0213063, and U.S. Pat. No. 5,538,898, the specifications and drawings of all of which incorporated by reference herein for all purposes). The emitter can be from the family of materials of carbon nanotubes, nanowires, carbene and the like. Molecular characteristic of a target agent can be a genetic characteristic such as a gene expression, a protein expression, and the like.

Molecular profile 16 can be generated by analysis instrument 14 as an outcome signal to the analysis system and its algorithms, such as analysis algorithm 18. In some embodiments, the molecular profile 16 is automatically and electronically transmitted from the instrument 14 to an interface or input of the analysis system 24, for example, through direct wireline or other electrical connection or via a communication network, such as any wired and/or wireless communication network or mechanism between the instrument 14 and the analysis system 24. In one embodiment, algorithm 18 is implemented as executable instructions stored on one or more memory devices and executed by one or more processors to implement the intended functionality. In some embodiments, the functionality of the analysis system is fully automated, while in other embodiments, the functionality is initiated by a user of the analysis system 24. The algorithm 18 can perform many functions depending on the type of molecular analysis desired and application or uses thereof. In one embodiment, the analysis algorithm 18 implements a searching algorithm to search one or more molecular profile databases (e.g., illustrated as database 20) to retrieve one or more comparative molecular profiles against which the molecular profile 16 can be compared, analyzed and/or correlated. Accordingly, in some embodiments, the algorithm 18 implements a comparison or correlation algorithm that can compare the output molecular profile 16 against comparative molecular profiles retrieved from the database 20. In some embodiments, the algorithm 18 implements a comparison of the molecular profile 16 against a predetermined database 20 of molecular profiles using a molecular profile algorithm and generates a report 22 that can have content information of relevance for a medical procedure or a forensic procedure. For example, the analysis system 24 can use an algorithm 18 (e.g., a statistical algorithm) to generate an information pattern that serves as an indication of a future biological event (expressed as either up or down regulated) correlated to the genetic characteristics.

It is noted that while all features of some embodiments of the present system are communicatively connected, many or even all of the features of the system can be combined into one physical unit and/or may not be communicatively coupled. This would be useful in, for example a forensics analysis that could allow an automated device to receive a specimen sample and generate a report. In some circumstances, the report could be generated remotely. The database 20 may also reside in a remote location, but accessible to the system by one or more networks, including various possible wired and/or wireless Internet, Intranet, local area network, wide area network or other network connections.

FIG. 2 illustrates a possible flow diagram of a method that can be used for one or more of the present embodiments. First, a specimen from an organism (subject) is provided at step 30 and collected onto a collector at step 31, e.g., using a collector substrate, such any of the collectors described herein or understood by those of ordinary skill in the art. Next, the collected specimen is transferred to an automated microdevice instrument at step 32, e.g., the specimen is introduced into an instrument such as analysis instrument 14. Generally, and in accordance with several embodiments, the automated micro device instrument processes the specimen and produces a molecular profile of the specimen in an automated manner. In accordance with one embodiment of this automated processing, reagents can be added to the specimen at step 34 to perform at least one bioassay reaction on the specimen at step 36. Next as a result of step 36, a specific and predetermined molecular species can be isolated from the specimen at step 38. In some embodiments, one or more of steps 34, 36 and 38 are performed by a specimen processor portion 60 of an analysis instrument. Next, at step 40, in some embodiments, the system can perform a molecular analysis or interrogation of the specimen that can include the steps of examining the species, detecting a specific signal output from that molecular species undergoing some bioassay reactions, which if detected by some detectable label, can generate a signal that can be processed. Then, a molecular profile is generated that is outputted to the analysis system (e.g., one embodiment of analysis system 24) at step 42. In some embodiments, one or more of steps 40 and 42 are performed by a specimen detector portion 62 of an analysis instrument. In some embodiments, the analysis instrument 14 of FIG. 1 can be involved in the performance of steps 32, 34, 36, 38, 40 and 42. In some embodiments, one or more of steps 32, 34, 36, 38, 40 and 42 are automated and performed under computer control initiated by a user at the instrument or by a user at the analysis system 24 or other remote user. Furthermore, in some embodiments, the molecular profile generated by step 42 is automatically and electronically transmitted from the specimen detector 62 to an interface or input of the analysis system 24, for example, through direct wireline or other electrical connection or via a communication network, such as any wired and/or wireless communication mechanism between the instrument 14 and the analysis system 24. It is understood that the analysis instrument 14 and the analysis system 24 can include the appropriate wireless, wired, and/or optical transmitting and receiving devices to effect the transfer of signaling therebetween and with the network 120. In other embodiments, the molecular profile is transferred to the analysis system 24 by a user, for example, using a portable digital media, such as a disk based or solid state based digital memory device.

Once the specimen's molecular profile is outputted from the automated instrument, the molecular profile can then be processed through one or more algorithms at step 44. In some cases, statistical algorithms are used whereas in other cases, non-statistical algorithms may be implemented, e.g., in cases where a simple match comparison of the molecular profile to one or more comparative molecular profiles is desired. In some embodiments, this process of step 44 can involve interrogating the specimen output against databases through data mining methods 46 to calculate comparison or statistical values at step 48 that can be assessed in quantity and threshold values of comparison and identity. Mined databases can reside on the system or be in third party databases or remote databases communicatively connected the system. In all aspects where the system communicates via networks and/or with third party databases or reporting, appropriate data security precautions should be taken and in some cases, are required to be taken depending on the sensitivity of the information in the databases or analysis. Predetermined thresholds can include predetermined degree or proportion of sequence identity, sequence partial identity, or substantial identity (see definitions above). The value at step 48 is next tested at step 49 as to whether it has reached an acceptable qualitative value based on a set of threshold values. The set of threshold values may be predetermined in advance of the analysis or dynamically determined based at least in part of the results of step 48. In one example, the system at step 49 comprising a qualitative comparative analysis of the molecular profile output of the test instrument with a stored pre-selected qualitative criteria profile, and outputting a feedback adjustment to the instrument when a predetermined qualitative threshold is not achieved by the molecular profile output. Thus, if the value of “no” is determined at step 49 (i.e., the predetermined quality or quantity threshold is not reached), the system can adjust the processing of the specimen at step 36 through a feedback mechanism at step 50 communicatively connected to the analysis instrument 14. At step 50, feedback signaling is communicated to the analysis instrument 14, so that the system is readjusted to adjust specimen processing values and re-process the specimen through the bioassay steps. However, if the value of “yes” is determined at step 49 (i.e., the predetermined or dynamically determined quality or quantity threshold is reached), the system can trigger a report at step 52 that can be configured to assist in determining options for a medical or forensic procedure or identification. Threshold determinations can be assisted by the use of DNA P-scores, such as those identified by the National Center for Biotechnology Information in its publication The Statistics of sequence Similarity Scores, which can be found at (http://www.ncbi.nlm.nih.gov/BLAST/tutorial/Altschul-1.html) and is incorporated herein by reference. For the present embodiments, the system can be calibrated in a DNA forensic application to set a threshold local P number that is considered below a threshold sensitivity to test accurately. If the threshold is not met, then the system can signal the instrument to adjust its procedures. This could include adding the steps to load more specimen from the collector.

In some embodiments, the report is stored by the analysis system 24 and may be communicated, e.g., by secure email, secure remotely accessed server or other data transmission mechanism to those that need to view the results of the analysis. In some embodiments, the report may be viewable on a remote computer, on a display of the analysis system 24, on a display of the instrument 14 or other display.

For ease in understanding the connectivity and functionality of some embodiments of the present system, FIG. 3 illustrates a system 100 according to several embodiments. As shown, system 100 includes an analysis instrument 14 comprising a specimen processor 102 and a specimen detector 104, an analysis system 24 executing an analysis application 106, and a local database 108. The system 100 also includes remote databases 110 and 112, remote computers 114, mobile device 116 and an email server 118 variously coupled to the analysis instrument 14 and/or the analysis system 24 via a network 120. The network 120 may be one or more of any wired and/or wireless point-to-point connection, local area network, wide area network, internet, intranet and so on.

In one embodiment, the specimen processor 102 of the analysis instrument 14 receives a biological specimen from a collector 12 (or extracted from collector 12). The specimen processor may include one or more specimen or sample processing techniques such as those described herein to prepare the specimen for detection or interrogation by the specimen detector 104. For example, the specimen processor 102 manipulates the specimen with reagents to ready the specimen for detection. In one embodiment, the specimen processor outputs the specimen and any biological and/or chemical data pertaining to the specimen to the specimen detector 104. The specimen detector 104 detects or interrogates the specimen or sample using one or more detection devices such as those described herein. In one embodiment, for example, the specimen detector 104 can capture fluorescence, radioactivity, color, chemiluminescence, and the like. In one embodiment, the specimen detector 104 outputs a molecular profile 16 to an input or interface 105 of the analysis system 24 and in another embodiment, the molecular profile 16 is output to the analysis system 24 via the network 120 and an interface 107 of the analysis system 24. It is understood that the interfaces 105 and 107 may be any wireless, wired or optical input that can interface with the corresponding device. For example, interface 107 may be a network interface. It is understood that the analysis instrument 14 and the analysis system 24 (e.g., the interfaces 105 and 107) can include the appropriate wireless, wired, and/or optical transmitting and receiving devices to effect the transfer of signaling therebetween and with the network 120 and any databases. In some embodiments, the analysis instrument 14 may be an automated and integrated device having both the specimen processor 102 and the specimen detector 104. In such integrated embodiments, the specimen is moved within the instrument 14 in a manner that is automated and not susceptible to contamination through handling. In other embodiments, the specimen processor 102 and the specimen detector 104 are not integrated and these functions may be performed by separate devices that may or may not be communicationally coupled together. For example, in one embodiment, the data output of the specimen processor 102 is electronically transmitted or delivered to the specimen detector 104 (directly via interface 105 or indirectly via the network 120 and interface 107), while the prepared specimen is physically transported (e.g., within a carrier or cartridge) to the specimen detector 104. In another embodiment, the data output of the specimen processor 102 is directly electronically transmitted or delivered to the specimen detector 104 (or processor controlling the specimen detector), while the prepared specimen is physically transported (e.g., within a carrier or cartridge) within the analysis instrument 14 to the portion functioning as the specimen detector 104. In some embodiments, the analysis instrument 14 is a processor-based device having at least one microprocessor, at least one memory device or computer readable medium, and executable program instructions stored by the at least one memory device or computer readable medium and executed by the at least one microprocessor to perform one or more functions, such as those described herein. Additionally, the hardware and software (or firmware) control components of the analysis instrument provide for the control of any included devices that process or detect the specimen.

In one embodiment, the analysis system 24 has stored thereon or stored in a memory or computer readable medium accessible by the analysis system software for executing the analysis application 106 for receiving and processing information and data from the analysis instrument 14, such as a molecular profile 16, and generating various outputs, such as report 22. In one embodiment, the application 106 further comprises one or more functional application components for processing the input from the analysis instrument 14, data mining, data comparisons, report generation, quality control checks, result reporting and/or transmitting adjustments back to one or both of the specimen processor 102 and the specimen detector 104. The output of the specimen detector 104 is provided at the input or the interface 105 of the analysis system 24. The interface may be a physical input, output or connector, or may be a functional interface implemented in received signaling in the control signaling or processor of the analysis system 24. The interface 105 may be a direct input from the specimen detector 104 (as illustrated) or may be the interface 107, e.g., a network interface that receives data or electronic signaling representing the molecular profile from the network 120.

In some embodiments, the specimen processor 102 and/or the specimen detector 104 are proximate to the analysis system 24. In other embodiments, the specimen processor 102 and/or the specimen detector 104 are remotely located from the analysis system 24, e.g., the specimen processor 102 and/or the specimen detector 104 are integrated into a field device that is local to the specimen collection location and the analysis system 24 is remotely located at a laboratory or other analysis location. In such case, the output of the specimen processor 102 and/or the specimen detector 104 is automatically and electronically delivered to the analysis system 24, e.g., using the network 120. This received data may be stored at the analysis system 24 or a local database 108 coupled thereto. In some embodiments, output from the analysis instrument 14 is stored at a remote computer 114 and/or a remote database 110 and/or 112, such that the information or output can be accessed by or retrieved by the analysis system 24 automatically or responsive to a user's instructions at the analysis system 24.

In one embodiment, the analysis system 24 comprises a computer or computer device including at least one processor, at least one memory or computer readable medium that stores and executes computer program code to implement its functionality. In one embodiment, the computer program code is remotely stored and executed for example, using a remote server or remote computer 114, and the analysis system 24 serves as the user interface. In one embodiment, the analysis system 24 is a single computer or computer device, whereas in other embodiments, the analysis system 24 is a plurality of computers or computer devices working together to implement the functionality of the analysis system.

In one embodiment, the computer or computer devices of the analysis system 24, the remote computers 114, and/or the mobile device 116 can be personal computers in communication with one or more other devices via the network 120 (i.e., any network accessible, enabled or addressable device, for example, in the case of the internet, a device having an IP address). Example computers or computer devices include but are not limited to desktop computers, laptop computers, personal data assistants (PDAs), smartphones, touch screen computing devices, handheld computing devices, or any other computing device having functionality to couple to the network 120. Additionally, the application 106 may also include a web application that acts to serve web content to other remote computers (e.g., remote computers 114, mobile device 116, etc.). These remote computes may include web browser capabilities and are able to access the web application component of the application 106 using a web browser to interact with and/or control one or more automated processes of the analysis system 24 and/or the analysis instrument 14.

In one or more embodiments, the system 100 includes remote databases 110 and 112 that may be accessed by the analysis system 24 or written to by the analysis system 24 and/or the analysis instrument 14. In some embodiments, databases 110 and 112 can be used to store predetermined databases for comparisons or correlations with data relating to collected specimens processed by the analysis instrument 14, such as the generated molecular profile 16. In some embodiments, such as for forensics identification, databases 110, 112 can be part of a third party database for comparison with or correlation to the collected specimen for identification. For example, database 110, 112 could be the Combined DNA Index System (CODIS). CODIS is a DNA database funded by the United States Federal Bureau of Investigation (FBI). The system that stores DNA profiles created by federal, state, and local crime laboratories in the United States, with the ability to search the database to assist in the identification of individuals. The DNA profile information stored in these databases, also known as a DNA type, is for Forensic STR DNA analysis the DNA profile of one or two alleles at the 13 CODIS core loci. Other potential databases 110, 112 could be used, such as those at the National Institute of Health (NIH) and specifically the NIH's databases with National Cancer Institute (NCI). Still others can include fisheries and or agricultural databases, such as those maintained by the National Oceanic and Atmospheric Administration. Further DNA profiles from the European Union's Prom database could also be considered as a source for data analysis as well as fisheries and agricultural databases such as those found with the National Oceanic and Atmospheric Administration (NOAA).

As described herein, in some embodiments, the analysis system 24 can generate a report relating to the analysis or correlation performed by the analysis system. In one embodiment, the report is displayed to a user at the analysis system 24. In another embodiment, the report is transmitted for display at the analysis instrument 14 (transmitted either directly or via the network 120). In another embodiment, the report is transmitted for display at one or more remote computers 114 and/or the remote device 116 via the network 120 the analysis instrument 14. In such case, the application 106 may serve the report in a web application for example using a markup language or web protocol format displayable at the remote computers 114 and/or the remote device 116, for example, using a web browser. Alternatively, the report may be emailed to the remote computer 114 and/or the remote device 116 using an email server 118 that is local to the analysis system 24 or communicationally coupled to the analysis system 24 via the network 120. Depending on the embodiment, the transmission of the report may be required to be through a secure network or routed through a non-secure network in a secure manner, e.g., using encryption or through a virtual private network (VPN) or other tunnel technology. Access to reports may additionally be password controlled.

In further embodiments, one or more functions of the analysis instrument 14 and/or the analysis system 24 may be initiated or triggered by a user remote from the analysis instrument 14 or analysis system 24. For example, a user at an authorized remote computer 114 or remote device 116 may be able to log in to one or both of the analysis instrument 14 and the analysis system 24 and remotely initiate, monitor, manage, terminate, etc. one or more functions of such devices.

In further embodiments, depending on the application, since the analysis system handles the molecular comparison or correlation of the molecular profile to the comparative molecular profile/s and provides output reports, the analysis system may be linked to other systems, such as other crime labs, databases, health care, insurance, etc., systems (generically illustrated as remote computers 114) via the network.

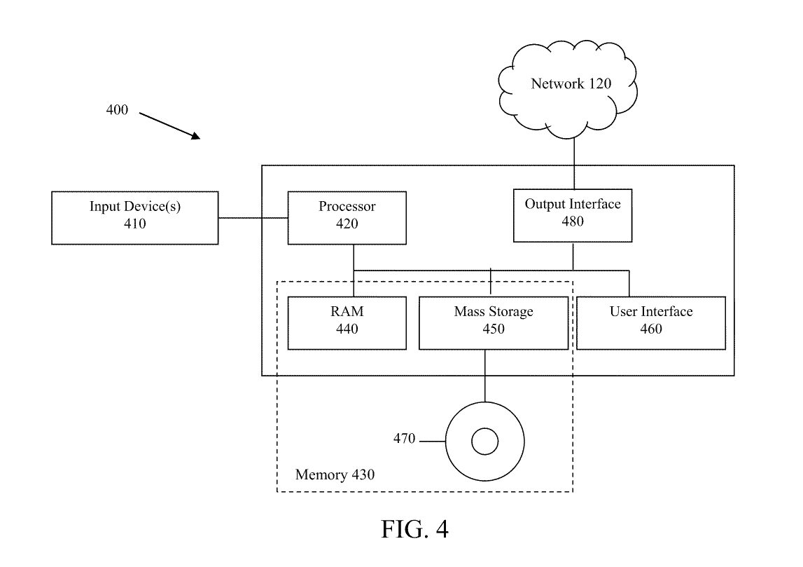

The methods and processes described herein may be utilized, implemented and/or run on many different types of systems. Referring to FIG. 4, there is illustrated a processor-based system 400 that may be used for any such implementations. One or more components of the system 400 may be used for implementing one or more of the control components of one or more of the analysis instrument 14, the analysis system 24, the remote computers 114 and the remote device 116. By way of example, the system may comprise at least one processor 420 (also referred to as a Central Processing Unit (CPU); a memory 430 (also referred to as a computer readable medium) generally including a Random Access Memory (RAM) 440, a mass storage unit 450 (such as a disk drive), and an external memory 470; and a user interface 460 such as a display. The processor 420 may be used to execute or assist in executing the steps of the methods and techniques described herein. The system may further comprise one or more input devices 410. The devices may comprise any user input device such a keyboard, mouse, touch screen keypad or keyboard. The input devices 410 may further comprise one or more processing and/or detection hardware components.

The mass storage unit 450 may also be referred as a memory and may include or comprise any type of computer readable storage or recording medium or media. The computer readable storage or recording medium or media may be fixed in the mass storage unit, or the mass storage unit may optionally include an external memory device 470, such as a digital video disk (DVD), Blu-ray disc, compact disk (CD), USB storage device, floppy disk, RAID disk drive or other media. By way of example, the mass storage unit 450 may comprise a disk drive, a hard disk drive, flash memory device, USB storage device, Blu-ray disc drive, DVD drive, CD drive, floppy disk drive, RAID disk drive, etc. The mass storage unit 450 or external memory device 470 may be used for storing executable program instructions or code that when executed by the processor, implements the methods and techniques described herein. Any of the applications and/or components described herein may be expressed as a set of executable program instructions that when executed by a processor (such as CPU), can performed one or more of the functions described in the various embodiments herein. It is understood that such executable program instructions may take the form of machine executable software or firmware, for example, that may interact with one or more hardware components or other software or firmware components.

Thus, external memory device 470 may optionally be used with the mass storage unit 450, and may be collectively referred to as the memory 430, which may be used for storing code that implements the methods and techniques described herein. However, any of the storage devices, such as the RAM or mass storage unit, may be used for storing such code. For example, any of such storage devices may serve as a tangible computer storage medium for embodying a computer program for causing a computer or display device to perform the steps of any of the methods, code, and/or techniques described herein. Furthermore, any of the storage devices, such as the RAM 440 or mass storage unit 450, may be used for storing any needed database(s). Furthermore, the system 400 may include external outputs at an output interface 480 to allow the system to output data or other information to other servers, network components or computing devices in the overall system 100 via one or more networks 120, such as described throughout this application.

While the figures and descriptions herein have been described in conjunction with specific embodiments, many alternatives, modifications, and variations will be apparent to those skilled in the art in light of the foregoing description. Changes in form, as well as substitution of equivalents, are contemplated as circumstances may suggest or render expedient.

What is claimed is:

1. A method for automated processing and analysis of a specimen from an organism, the method comprising:
receiving a biological specimen into an automated and integrated instrument, the biological specimen released from a collector that selectively collects species from the biological specimen;
isolating at least one molecular species representing at least one set of genetic characters of the specimen;
supplying at least one reagent to perform an assay on the at least one molecular species to detect at least one representative expression phenomenon of genetic characters;
monitoring at least one change in the at least one representative expression phenomenon during the assay;
translating the genetic characters into a molecular profile; and
analyzing the molecular profile using at least one of a statistical and mathematical algorithm to generate an information pattern that serves as an indication of a future biological event correlated to the genetic characters.

2. The method of claim 1, wherein the step of measuring the molecular data using a statistical algorithm comprises the steps of:
mining databases for comparative molecular profiles; and
comparing the molecular profile of the specimen with a comparative molecular profile from the database.

3. A rapid diagnostic method for a human specimen, comprising the steps of:
providing a specimen to a mobile automated integrated device capable of analysis of specimen preparations having fluid volume as small as 1 fl, low target concentrations of as little as 1 fmol, and small features of as small as 1 nm;
processing the specimen to prepare a target agent for at least one of microscopic, genetic, proteomics and genomic analysis;
interrogating the target agent;
collecting at least one of a morphological and molecular characteristic of the target agent;
comparing the characteristic of the target agent with a reference agent;
analyzing a difference between target and reference agents to provide information about at least one of a morphology, genome, proteome, and metabolome of the target agent;
generating a data output; and
outputting the data output to a computer via a communication network.

4. The method according to claim 3 wherein the specimen is a nucleic acid.

5. The method according to claim 3 wherein the integrated device comprises a microfluidic channel network.

6. The method according to claim 3 wherein the target agent is a viral pathogen.

7. The method according to claim 3 wherein the target agent is a bacterial pathogen.

8. The method according to claim 3 wherein the target agent is a spore.

9. The method according to claim 8, wherein the spore is from an anthracis species.

10. The method according to claim 3, wherein an analysis comprises a power light emitter, optical means to guide radiation from a radiation source and collect radiation from the target agent when interrogated by the source, a detector, and a device for microscopic analysis.

11. The method according to claim 10, wherein the emitter is from the family of materials selected from the list of carbon nanotubes, nanowires and carbene.

12. The method according to claim 3, wherein the molecular characteristic of the target agent is a genetic characteristic selected from one of a gene expression and a protein expression.

13. A system to provide automated and integrated matching of a biological specimen to an individual, comprising:
a collector having substrate materials and configured to selectively collect and release at least one bio-molecular species from a biological specimen;
a self-contained mobile automated testing instrument configured to receive the biological specimen and further configured to generate, store and output a molecular profile of the at least one predetermined bio-molecular species;
a processor based system communicatively coupled to the self-contained mobile automated testing instrument and configured to receive the molecular profile; and
wherein the processor based system is configured to provide a quantitative comparative analysis and report of the molecular profile with a selected molecular profile.

14. The system of claim 13, wherein the at least one predetermined bio-molecular species and stored pre-selected molecular profile is for a predetermined sequence of interest.

15. The system of claim 14, wherein the sequence of interest is a DNA-profile as defined by a Combined DNA Index System (CODIS) system.

16. The system of claim 15, wherein the DNA-profile is at least one allele at the 13 CODIS core loci for forensic STR DNA analysis.

17. The system of claim 13, wherein the processor based system is further configured to provide a qualitative comparative analysis of the molecular profile output from the test instrument with a stored pre-selected qualitative criteria profile, and configured to output a feedback adjustment to the self-contained mobile automated testing instrument when a predetermined qualitative threshold is not achieved.

18. The system of claim 17, wherein the feedback adjustment adjusts a bioassay process of the self-contained mobile automated testing instrument.

19. The system of claim 13, further comprising a memory of the processor based system, wherein the pre-selected molecular profile is stored on the memory.

20. The system of claim 13, further comprising a third party database communicatively coupled to the processor based system wherein the pre-selected molecular profile is stored on the third party database.

21. A computer system to provide automated analysis of molecular profiles of biological specimens, the computer system comprising:
an interface configured to receive a molecular profile of a biological specimen collected with a collector and interrogated to result in the molecular profile;
at least one processor;
at least one memory storing executable program instructions,
wherein the processor is programmed, via execution of the executable program instructions, to:
perform an analysis of the molecular profile relative to at least one comparative molecular profile retrieved from a database; and
provide a report based on the analysis.

22. The computer system of claim 21, wherein the analysis comprises a quantitative comparative analysis.

23. The computer system of claim 21, wherein the analysis comprises a quantitative statistical comparative analysis.

24. The computer system of claim 21, wherein the processor is programmed, via execution of the executable program instructions, to:
determine database search parameters corresponding to the molecular profile: and
retrieve the at least one comparative molecular profile from the database based on the database search parameters.

25. The computer system of claim 21, wherein the processor is programmed, via execution of the executable program instructions, to provide the report for display at a remote computer device.

26. The computer system of claim 21, wherein the processor is programmed, via execution of the executable program instructions, to output feedback adjustment signaling to an instrument configured to process the biological specimen as a part of the process of creating the molecular profile.

27. The computer system of claim 21, further comprising an analysis instrument configured to:
process the biological specimen for detection;
detect a genetic characteristic of the biological specimen;
generate the molecular profile; and
output the molecular profile to the interface of the processor.

28. The computer system of claim 27 wherein the analysis instrument is communicatively coupled to the interface of the processor.

29. The computer system of claim 27 wherein the analysis instrument is communicatively coupled to the interface of the processor via a network.

30. The computer system of claim 21 wherein the processor is configured to perform the analysis and provide the report in an automated manner upon receipt of the molecular profile.

31. A computer implemented method for automated analysis of molecular profiles of biological specimens, the method implemented with at least one processor executing program instructions stored in at least one memory, the method comprising;
receiving, via a computer system interface, a molecular profile of a biological specimen collected with a collector and interrogated to result in the molecular profile;
retrieving at least one comparative molecular profile from a database;
performing, using the at least one processor, an analysis of the molecular profile relative to the at least one comparative molecular profile; and
providing, using the at least one processor, a report based on the analysis.
